(19) United States
(12) Patent Application Publication
(10) Pub. No.: US 2014/0371084 A1
(43) Pub. Date: Dec. 18, 2014

(54) SNRNA RNU2-1 AS A TUMOR MARKER

(71) Applicants: Wolff Schmiegel, Bochum (DE); Alexander Baraniskin, Dortmund (DE); Stephan Hahn, Bochum (DE)

(72) Inventors: Wolff Schmiegel, Bochum (DE); Alexander Baraniskin, Dortmund (DE); Stephan Hahn, Bochum (DE)

(21) Appl. No.: 14/234,267

(22) PCT Filed: Jul. 20, 2012
(86) PCT No.: PCT/EP2012/064321

§ 371 Date: Aug. 29, 2014

(30) Foreign Application Priority Data
Jul. 22, 2011 (DE) 10 2011 108 254.2
Mar. 16, 2012 (DE) 10 2012 005 153.0

(51) Int. Cl. C12Q 1/68

(52) U.S. Cl. CPC C12Q 1/6886
USPC 506/9; 536/24.31; 536/24.33; 435/6.14; 435/6.12; 435/6.11

(57) ABSTRACT

The invention relates to a method for the diagnosis of malignant diseases comprising the steps
Provision of a sample obtained from a bodily fluid or bodily excretion as well as
Determination of the concentration of snRNA RNU2-1 or their fragments in the sample,
   as well as a kit for the performance of this diagnosis, and the use of probes specific to RNU2-1 and fragments thereof.

The invention relates to a method for diagnosis of malignant diseases, with said method being based on the determination of the content/level of a certain snR-NA, RNU2-1f or partial sequences thereof found in various bodily fluids, as well as to a kit for establishing a diagnosis of such diseases and using this snRNA and its partial sequences and primers specific thereof to diagnose such diseases.

Small nuclear RNAs (snRNAs) are small RNA molecules involved in a number of important processes. As a constituent of the spliceosome snRNAs are catalytically active. They are responsible for the detection and splicing of the intron of the pre-mRNA present in the cell nucleus. Moreover, they form complexes with several specific proteins, so-called snRNPs (small nuclear ribonucleoprotein particles) or snurps.

Using RNA or DNA amplification techniques snRNAs and their partial sequences are detectable in various bodily fluids such as blood or serum/plasma, ascites, pleural effusion, liquor cerebrospinalis, and urine. Specific expression patterns of snRNAs can be found in various types of tumors.

No investigations have hitherto been reported with respect to snRNAs RNU2-1 und their partial sequences in serum/plasma, ascites, pleural effusion, liquor cerebrospinalis, pancreatic duct secretion, pancreatic cyst fluid, vitreous humor, bile, stool, or urine of patients suffering from neoplastic diseases.

Following circulatory diseases, malignant neoplasms continue to be the second most common cause of death for both sexes in Germany despite significant progress that has been achieved in the field of oncology in recent decades. At the present time, approximately one person in four dies as a result of cancer. Tumor markers primarily used so far for the assessment of the disease history are endogenously developing substances which in some tumor diseases are increasingly present in the blood. It is the tumor cells themselves that form these substances or incite their formation.

It would, therefore, be desirable to have available a tumor marker which with high sensitivity and specificity finds evidence suggesting the presence of a malignant disease. In particular, there is need for a tumor marker that independently of the tumor type delivers conclusive data and can be used for differential diagnostic purposes.

Surprisingly, it has now been found that the snRNA RNU2-1 and in particular its fragments [herein referred to as RNU2-1f], also vesicularly protected fragments of the RNU2-1 isolated from bodily fluids, constitute such a tumor marker which can be detected with relatively high sensitivity and/or specificity and enables a conclusive diagnosis to be reached with respect to a patients tumor situation.

Accordingly, the invention relates to a method for the diagnosis of malignant diseases and comprises the following steps:
Provision of a sample obtained from a bodily fluid or bodily excretion as well as
Determination of the concentration of the snRNA RNU2-1 or partial sequences thereof.

The snRNA which according to the invention serves as marker has the following nucleotide sequence:

RNU2-1
SEQ0
ATCGCTTCTC GGCCTTTTGG CTAAGATCAA GTGTAGTATC
TGTTCTTATC AGTTTAATAT CTGATACGTC CTCTATCCGA
GGACAATATA TTAAATGGAT TTTTGGAGCA GGGAGATGGA
ATAGGAGCTT GCTCCGTCCA CTCCACGCAT CGACCTGGTA
TTGCAGTACC TCCAGGAACG GTGCACCC
(NCBI Reference Sequence RNU2-1: No. 0027 16.3)

Diagnostically conclusive fragments (RNU2-1f) of RNU2-1 are listed hereunder:

| SEQ 1: 5′-...CAATATATTA AATGGATTTT TGGAGCAGGG | | | | |
|---|---|---|---|---|
| Sm site | | | | |
| AGATGGAATA GGAGCTTGCT CCGTCCACTC CACGCATCGACCT...-3′ | | | | |
| SEQ 2: | AATGGATTTTTGGAGCA | 1× | | |
| SEQ 3: | AATGGATTTTTGGAGCAG | 1× | {close oversize brace} | 24% |
| SEQ 4: | AATGGATTTTTGGAGCAGG | 29× | | |
| SEQ 5: | AATGGATTTTTGGAGCAGGG | 54× | | |
| SEQ 6: | AATGGATTTTTGGAGCAGGGA | 8× | | |
| SEQ 7: | AATGGATTTTTGGAGCAGGGAG | 31× | | |
| SEQ 8: | AATGGATTTTTGGAGCAGGGAGA | 2× | {close oversize brace} | 76% |
| SEQ 9: | AATGGATTTTTGGAGCAGGGAGAT | 1× | | |
| SEQ10: | AATGGATTTTTGGAGCAGGGAGATGG | 2× | | |
| | | 129 | | |

In its nucleus snRNA RNU2-1 has a nucleotide sequence that (aside from other sequences) furnishes relevant information about the tumor situation. This nucleotide sequence has been shown in bold face in SEQ1 and listed in the above table as SEQ4.

In all nucleotides the nucleotide succession of SEQ4 corresponds to a MicroRNA miR-1246 which forms part of the nucleotide sequence pre-miR-1246. However, this pre-miR-1246 has nothing to do with tumor incidences of interest in this context; the coincidence with the central nucleotide sequences is purely accidental.

Nevertheless, for the purpose of the invention the diagnostic procedure developed for miR-1246 can be employed to offer proof of tumor activities in patients.

Fragments SEQ2 to SEQ10 mentioned hereinbefore are sequences derived from QIAGEN FOR products and can be used for tumor diagnosis purposes. In comparison with sequence SEQ4 the sequences SEQ2 and SEQ3 are by one and respectively two nucleotides shorter at the 3′ end while the sequences SEQ5 to SEQ10 include nucleotides in excess of those in SEQ4 and, therefore, are suited for a differentiation versus miR-1246.

In the framework of cloning PCR products by way of the Qiagen miR-1246 qRT-PCR assay the above listed sequences SEQ2 to SEQ10 are found with the frequency of occurrence indicated. With 76% of the sequences a differentiation versus miR-1246 can be made while 24% of the fragments are sequences that coincide both with the miR-1246 and RNU2-1 sequence and, respectively, with partial sequences of both transcripts so that an unambiguous differentiation of both transcripts is not possible. (Own experimental data substantiate, however, that miR-1246 is not formed in human cells, and on this basis the interpretation can be derived that all the sequences listed (SEQ2-10) are to be regarded as fragments of the RNU2-1.)

In the framework of a research project the material (serum, blood, ascites, pleural effusion, liquor cerebrospinalis, bile and pancreatic duct secretions, sputum, vitreous fluid, pericardial effusion, and urine) obtained as a liquid phase by centrifugation of the cellular components was examined molecular genetically with respect to the expression of various snRNAs. In all substances snRNAs were detectable by means of quantitative real-time polymerase chain reaction (qRT-PCR), and the relationship between the expression of certain snRNAs in the bodily fluids listed and the diagnosis of the malignant tumor could be demonstrated successfully. It has been shown that a significant correlation existed between the abundance/concentration (overexpression) of fragments of the RNU2-1 in various bodily fluids and the most frequently occurring malignancies such as breast cancer, bronchial cancer, colorectal cancer, prostate cancer, pancreatic cancer, gastric cancer, ovarian cancer, lymphomas, cholangiocarcinorna, hepatocellular carcinoma, renal cell and bladder cancer as well as GIST. This enabled a distinction to be made not only between healthy and diseased patients but also between patients suffering from malignancies and those affected by differential diagnostically relevant inflammatory changes. For example, the examination of the serum of patients suffering from pancreatic cancer and colorectal cancer revealed a high specificity of 97.7 and, respectively, 90.6%.

Using the biomarkers SEQ1 as well as SEQ2 to SEQ 10 is not only limited to the initial and early diagnosis of malignant diseases. As is evident from the experiments they may also be employed as disease course markers and predictive markers. It has been found that the expression of fragments SEQ2 to SEQ10 correlated with the course of the disease (course marker) in case of colorectal carcinoma, bronchial and pancreatic cancer, with the concentration of these fragments decreasing with a good clinical response to chemotherapy. With this in mind, the inventive method is also of value for the treatment of malignant diseases. Moreover, it has been found that, for example, in the case of colorectal carcinoma and ovarian carcinoma the concentration of RNU2-1f, respectively the changing concentration of the marker molecule during therapy correlated with the time until progress or with survival.

The results obtained show clearly that the snRNA RNU2-1, respectively its fragments in the bodily fluids examined are to be regarded as a promising pan tumor marker for diagnostic purposes and to assess the progression of a malignancy. So far, no pan tumor markers have been described that possess similar sensitivity or specificity for the broad range of tumor entities.

The method according to the invention provides for the concentration to be determined of at least one fragment of the snRNA RNU2-1 which is diagnostically conclusive for the determination of whether a malignant disease of a patient exists or not. A qualitative determination can be performed with the help of a probe specific to miR-1246 (SEQ4). Due to the fact that minor amounts of RNU2-1 and its fragments are also detectable in healthy patients the content detected is compared with a reference value. On the one hand, such a reference value may be a standard value, with the value detected being compared to the value of a healthy patient (standard value), but as a rule is a relative value because such a value can be determined more easily. Such relative quantifications are basically known and require a reference value. Such a reference value may be the expression of an uninfluenced micro-RNA, that is a micro-RNA which is expressed is unchanged in healthy and diseased patients, but may also be an added synthetic micro-RNA the relative concentration of which in the sample is known. Such a synthetic micro-RNA, for example, is syn-cel-54 which has been developed as miRNA-mimic for reference purposes of this nature. Relative quantifications may, for example, be carried out in the form of quantitative real-time PCR (qRT-PCR).

Expediently, the total content of RNAs in a sample, where appropriate after amplification, is determined by fluorescence measurement. Quantitative determinations are made by way of primers specific to the respective RNA fragments, that is, via primers specific to miR-1246 (SEQ4) or via a primer specific to at least one of the sequences SEQ1 to SEQ10.

Quantitative determinations may also be performed by means of primers specific to other fragments than SEQ2 to SEQ10 of RNU2-1. It is advantageous in this instance as well if such a primer covers several fragments that are to be seen similar with respect to the succession of some nucleotides as is the case in SEQ1 to 10.

The invention also relates to a kit for the diagnosis of malignant diseases, said kit comprising at least one probe and/or one primer for the detection of snRNA RNU2-1, the SEQ1 and/or at least one of the fragments SEQ2 to SEQ10 of snRNA RNU2-1. Moreover, the kit expediently contains a reference RNA including pertinent probe and/or pertinent primer.

In conclusion, the invention also embraces the use of RNU2-1 and/or at least one of its fragments for the diagnosis of malignant diseases as well as the use of probes and/or primers specific to these fragments. In particular, the probes are also primers specific to miR-1246 (SEQ4), i.e. oligonucleotides hybridizing to it.

The invention particularly enables a differential diagnosis to be made to distinguish between non-malignant diseases and neoplasia. In the samples of patients suffering from a cancer disease an overexpression of RNU2-1 was detected via its fragments SEQ1 and SEQ2 to SEQ10 in comparison with the sample of a patient not suffering from cancer or of a healthy test person.

Techniques required in the framework of the inventive method to enrich/amplify the RNU2-1 fragments via primers to enable a qualitative and quantitative detection of the fragments as well as purification and contents determination are generally known.

The invention is explained in more detail by way of the following examples.

### SAMPLE PREPARATION

Samples (serum, whole blood, ascites, pleural effusion, urine, and liquor cerebrospinalis) were centrifuged within 60 min of sample taking (500×g, 10 min, RT) and the supernatant stored at −80° C.

RNA Extraction

From the material obtained the entire RNA contents was extracted with the aid of a miRVana RNA isolation kit (Ambion, Austin, USA) following the instructions issued by the manufacturer. For this purpose, deep-frozen samples were thawed on ice and 0.17 ml thereof diluted with the identical volume of miRVana PARIS 2× denaturing solution and subsequently incubated on ice for 5 min. Following this and for the purpose of normalizing, 5 μl each of synthesized miRNA-mimic syn-cel-54 of a concentration of 5 fmol/ml was added to the samples. Identical volumes of acid/phenol/chloroform (Ambion) were added to each aliquot and the samples subsequently centrifuged for 5 min. at 10,000×g. Glycogen was added to the aqueous phase, following which 1.25 volumes of 100% ethanol were admixed. After passage through a miRVana PARIS column several washing steps were carried out in accordance with the instructions prescribed by the manufacturer. Finally, the RNA was eluted in 100 μl of nuclease-free water. The RNA concentration was determined using a NanoDrop^{R} ND-3300 fluorospectrometer by way of 2 μl aliquot measurement.

Determination of miRNA Expression through Quantitative Real-Time Polymerase Chain Reaction

In conformity with the manufacturer's instructions Qiagen miRNA assays (Qiagen, Hilden, Germany) were made use of for the quantification of the microRNA levels: 2 μl of the entire RNA solution were employed for the reverse transcription reaction (37° C. for 60 min, and 95° C. for 5 min., followed by 4° C.). The real-time qPCR was performed by means of an Opticon 2 System (MJ Research, Waltham, Mass.) according to the protocol of the manufacturer (Qiagen, Hilden, Germany). The general cycle conditions were as follows: 95° C. for 15 min., 40 cycles of 15 s at 94° C., 30 s at 55° C. and 30 s at 70° C. Each sample was analyzed at least twice. For all micro-RNAs average threshold cycle values (Ct values) and standard desamples were calculated. The concentration of syn-cel-54 in the relevant was measured as well. The amount of target micro-RNA was normalized relative to the amount of syn-cel-54.

In accordance with data available at www.mirbase.org miR-1246 possesses the following nucleotide sequence:

miR-1246
AAUGGAUUUUUGGAGCAGG

This sequence coincides precisely with fragment SEQ4 of RNU2-1 and is contained almost completely or completely in the fragments SEQ1 as well as SEQ2, 3 and 5 to 10.

The expression profiles of patients suffering from the following diseases showed an RNU2-1 overexpression determined on the basis of fragments SEQ2 to SEQ10:
Neoplasia:
Pancreatic cancer
Colorectal cancer
HCC
CCC
Lung cancer
Renal cell carcinoma
Bladder cancer
Non-Hodgkin lymphoma
Primary CNS lymphoma
Ovarian cancer
Breast cancer
Prostate cancer
Gastric cancer
Pleural mesothelioma
Bile duct carcinoma
GIST
Sarcomas
Peritoneal mesothelioma
Diseases that may give rise to differential diagnosis:
Bacterial peritonitis
Pneumonia
Silicosis
COPD
Liver cirrhosis
Hepatitis
Cardiac decompensation
Pancreatitis
IBD
Collagenosis
Sarcoidosis

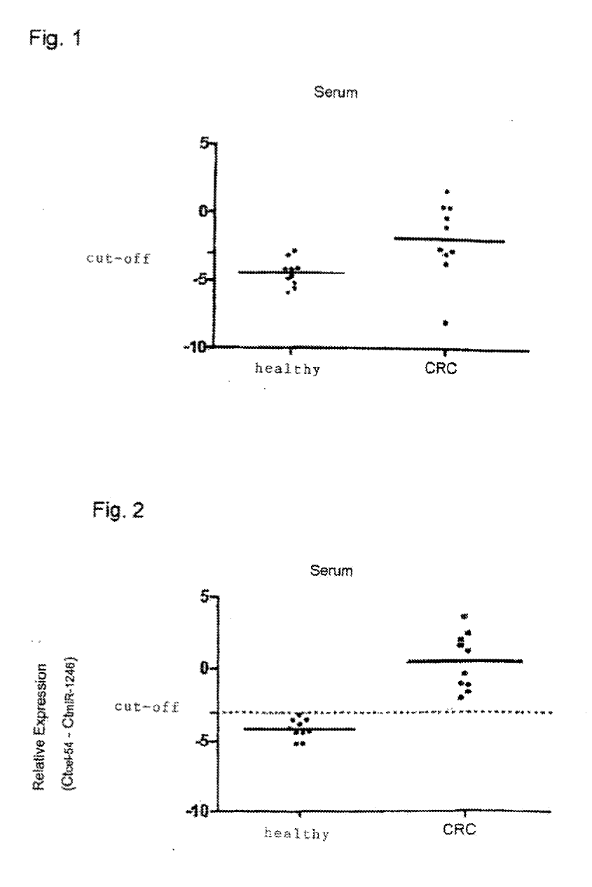

FIG. 1 shows the result of a serum test of a healthy patient and of a patient suffering from colorectal carcinoma, for which an assay was employed specific to the fragments SEQ4 to SEQ10.

FIG. 2 shows a corresponding test performed using the miR-1246 assay of Qiagen. The assay employed in FIG. 1 is specific with respect to the fragments of RNU2-1 but on the whole less sensitive. Due to the fact that the Qiagen assay for miR-1246 embraces more fragments this leads to a specificity increase.

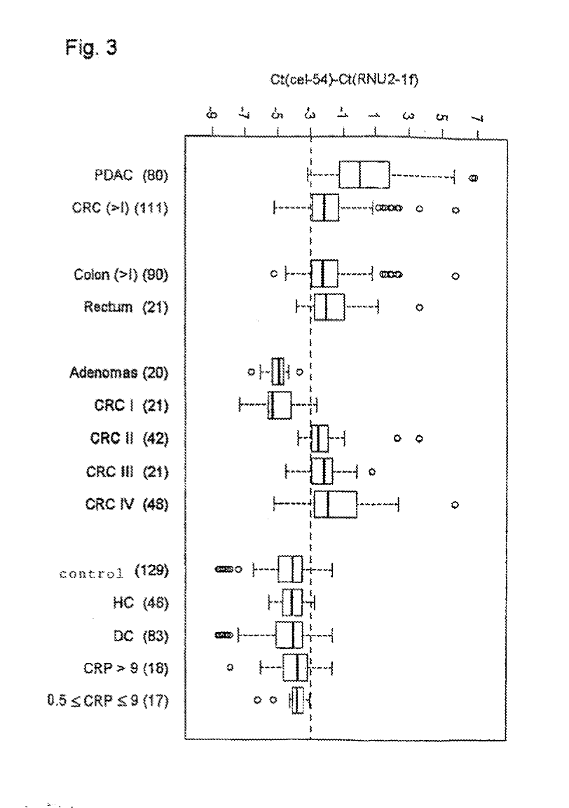

FIG. 3 shows the result of serum tests applicable to various malignant diseases and controls. The threshold value (cut off) is −2.995 and is illustrated by the broken line, PDAC stands for pancreatic ductal adenocarcinoma, CRC for colorectal carcinoma, R for rectal carcinoma, CRC I to IV for the UICC stages I to IV of the CRC, HC for healthy controls, DC for diseased controls (differential diagnoses), CRP for c-reactive protein.

1. Method for diagnosis of malignant diseases comprising the steps
Provision of a sample obtained from a bodily fluid or bodily excretion as well as
Determination of the concentration of snRNA RNU2-1 and/or their fragments in the sample.

2. Method according to claim 1, characterized by the comparison of the level of at least one of the fragments SEQ1 and/or SEQ2 to SEQ10 RNU2-1 with a threshold value.

3. Method according to claim 1, characterized by a relative quantification of the level of at least one of the fragments SEQ2 to SEQ10 with a comparison RNA.

4. Method according to claim 3, characterized in that the comparison RNA is a synthetic micro-RNA.

5. Method according to claim 1, characterized by the step of enriching the fragments SEQ2 to SEQ10 in the sample by real-time qPCR.

6. Method according to claim 5, characterized by a determination of the total RNA contents by fluorescence measurement.

7. Method according to claim 1, characterized by the step of quantitative determination of the fragments SEQ2 to SEQ10 via specific primers.

8. Method according to claim 1, characterized in that the patient sample is obtained from whole blood, serum, plasma, sputum, ascites, pleural effusion, stool, pancreatic secretion, bile duct secretion, urine, cystic fluid or liquor cerebrospinalis.

9. Kit for the diagnosis of malignant diseases, comprising at least one probe and/or one primer for the detection of at least one, in particular all of the fragments SEQ2 to SEQ10.

10. Kit according to claim 9, comprising a reference RNA together with the pertinent probe and/or pertinent primer.

11. Kit according to claim 10, characterized by at least one oligonucleotide hybridizing with SEQ4 (miR-1246), in particular with all the fragments SEQ2 to SEQ10 and the reference micro-RNA.

12. Use of probes specific to SEQ4 (miR-1246) for the diagnosis of cancer diseases.

13. Use according to claim 12, characterized in that the probe is an oligonucleotide hybridizing with SEQ4 (miR-1246).

14. Use according to claim 12, characterized in that the diagnosis includes a quantitative determination of the content of fragments SEQ2 to SEQ10 with the probe.

15. Use of the snRNA RNU2-1, fragments thereof and preferably at least one, in particular the entirety of the fragments SEQ2 to SEQ10 for the diagnosis of cancer diseases.


SEQUENCE LISTING (11 sequences)

SEQ ID NO: 1 - DNA, 187 bp, Homo sapiens
atcgcttctcggccttttggctaagatcaagtgtagtatctgttcttatcagtttaatatctgatacgtcctctatccgaggacaatatattaaatggatttttggagcaggagatggaataggagcttgctccgtccactccacgcatcgacctggtattgcagtacctccaggaacggtgcaccc

SEQ ID NO: 2 - DNA, 73 bp, Homo sapiens
caatatattaaatggatttttggagcagggagatggaataggagcttgctccgtccactccacgcatcgacct

SEQ ID NO: 3 - DNA, 17 bp, Homo sapiens
aatggatttttggagca

SEQ ID NO: 4 - DNA, 18 bp, Homo sapiens
aatggatttttggagcag

SEQ ID NO: 5 - DNA, 19 bp, Homo sapiens
aatggatttttggagcagg

SEQ ID NO: 6 - DNA, 20 bp, Homo sapiens
aatggatttttggagcaggg

SEQ ID NO: 7 - DNA, 21 bp, Homo sapiens
aatggatttttggagcaggga

SEQ ID NO: 8 - DNA, 22 bp, Homo sapiens
aatggatttttggagcagggag

SEQ ID NO: 9 - DNA, 23 bp, Homo sapiens
aatggatttttggagcagggaga

SEQ ID NO: 10 - DNA, 24 bp, Homo sapiens
aatggatttttggagcagggagat

SEQ ID NO: 11 - DNA, 26 bp, Homo sapiens
aatggatttttggagcagggagatgg